(19) United States
(12) Patent Application Publication
(10) Pub. No.: US 2018/0280197 A1
(43) Pub. Date: Oct. 4, 2018

(54) HEAD WORN DEVICES HAVING A VARIABLE SHADE COMPONENT AND FIXED SHADE COMPONENT, AND METHODS OF OPERATION

(71) Applicant: Illinois Tool Works Inc., Glenview, IL (US)

(72) Inventors: Nishank R. Patel, Appleton, WI (US); Eric Sommers, Appleton, WI (US)

(21) Appl. No.: 15/474,381

(22) Filed: Mar. 30, 2017

(51) Int. Cl. A61F 9/06

(52) U.S. Cl. CPC A61F 9/067

(57) ABSTRACT

Head worn devices having a variable shade component and a fixed shade component, and methods of operation, are disclosed. An example head worn device includes an auto-darkening lens configured to adjust a shade value within a first shade value range, a second lens having a fixed shade value greater than 0, the auto-darkening lens and the second lens overlapping in a field of view of the head worn device to result in a net shade value range in the field of view that is greater than the first shade value range, and a clear lens on an exterior of the auto-darkening lens and the second lens.

### BACKGROUND

Conventional welding helmets having auto-darkening lenses that automatically adjust between light and dark states to improve the welder's visibility when not welding and providing eye protection from arc light while welding. Depending on environmental or ambient lighting, welders may still have difficulty seeing through a heavily tinted lens and may remove or lift their helmets to get a clearer view. This action lowers their productivity and also may place more strain on the welder's neck due to the repeated rotation of the visor.

### SUMMARY

Head worn devices having a variable shade component and fixed shade component, and methods of operation are disclosed, substantially as illustrated by and described in connection with at least one of the figures, as set forth more completely in the claims.

### BRIEF DESCRIPTION OF THE DRAWINGS

FIG. 1 illustrates an example combination of a variable shade component and a fixed shade component for use in a head worn device, in accordance with aspects of this disclosure.

FIG. 2 illustrates installation of the fixed shade component of FIG. 1 into a lens retention device.

FIG. 3 illustrates combinations of an auto-darkening lens and a fixed shade lens and resulting net shade value ranges.

FIG. 4 is a flowchart representative of a method to operate the example head worn device of FIG. 1.

FIG. 5 illustrates another example head worn device in which a fixed shade lens may be moved into and at least partially out of a field of view of a variable shade lens.

FIG. 6 illustrates another example installation of the fixed shade lens of FIG. 1 into a lens retention device.

FIG. 7 illustrates another example combination of the variable shade component and the fixed shade component for use in a head worn device.

The figures are not necessarily to scale. Where appropriate, similar or identical reference numbers are used to refer to similar or identical components.

### DETAILED DESCRIPTION

Some conventional auto-darkening lenses have a lower shade range limit of 2.5, to improve wearer visibility when not welding. However, such lenses also limit the dark state of the lens to an upper shade limit of 12.5. Welding at high amperages and around reflective surfaces may require higher shade values due to the intensity of the welding arc light being very bright. A dark shade of 13.0 or more may be required maintain adequate eye protection according to ANSI and CE standards.

Additionally, a single shade range provided by an auto-darkening lens may not be appropriate for all welding applications encountered by a welder. Disclosed example head worn devices provide for easy adjustment of a shade range to enable operators to adapt the net shade range to different applications. For example, a head worn device may be configurable to provide a light state of 2.5 that increases visibility, and enable configuration of the shade range shade 13.0 if required by a specific welding application. Disclosed examples enable this adjustment without adding substantial or, in some cases, any additional weight to the welding helmet.

Disclosed example head worn devices use a fixed shade lens to modify a variable shade lens, such as an auto-darkening lens. Disclosed examples enable an operator to quickly modify a fixed shade lens on the head worn device to increase or decrease the net shade value range of the head worn device. The net shade value range is a combination (e.g., a sum) of the shade value of the fixed shade lens and the shade value range of the auto-darkening lens. Disclosed examples enable a welder to have multiple shade ranges without purchasing and maintaining multiple head worn devices. Instead, the welder may have multiple fixed shade lenses that are interchangeable in the head worn device to change the net shade value range.

Disclosed head worn devices be implemented as welding helmets, flip-up welding helmets with clear grinding shields, a welding helmet integrated with a cap, welding goggles, or welding glasses.

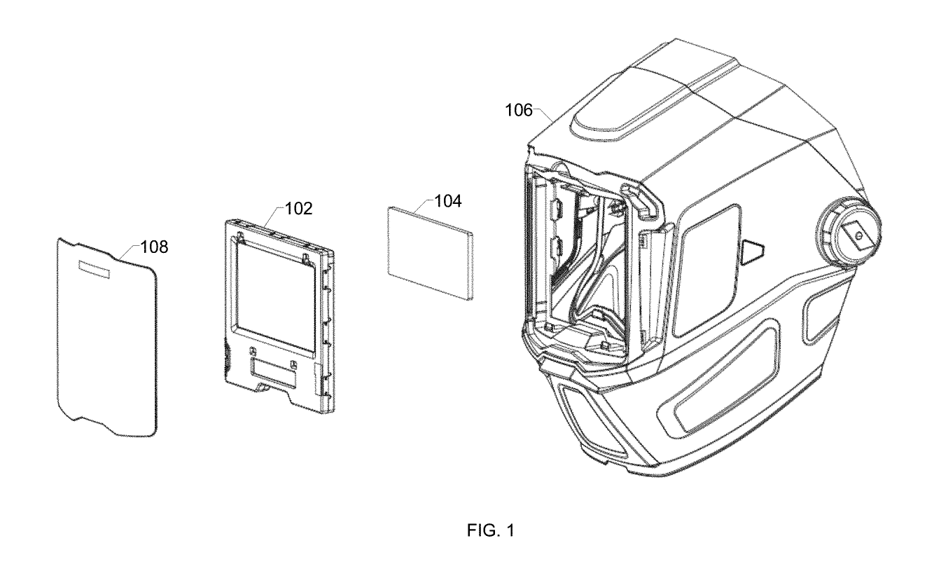

FIG. 1 illustrates an example combination of a variable shade component 102 and a fixed shade lens 104 for use in a head worn device 106. The example variable shade component 102 may be implemented using an auto-darkening lens. Auto-darkening filter lenses (ADF) use layers of polarizers and liquid crystals to filter out varying amounts of light based on electric input. In an inactive state (e.g., a light state), the auto-darkening lens may be set somewhere between a shade 3.0-5.0. As used herein, shade levels are used as defined in the standard ANSI Z87.1. However, other shade standards may be used. Each increasing value of shade level means a higher percentage of light is filtered. When a welder strikes an arc, an auto-darkening lens may darken to a selected higher shade range from 8.0-13.0 to protect the welder's eyes. The light state is used for non-welding situations, such as grinding and welding setup activities, and may be between shades 3.0-5.0.

The variable shade component 102 and the fixed shade lens 104 are positioned in a field of view of a wearer of the head worn device 106, and provide protection from high intensity light, such as arc light that occurs during welding. The variable shade component 102 changes a shade value within a shade value range. Example shade value ranges include shade values between 2.5-12.5 or shade values between 3.0-13.0. The variable shade component 102 changes the shade value based on, for example, an ambient light value (e.g., whether a welding arc is present).

In some examples, the fixed shade lens 104 is inexpensive to manufacture (e.g., relative to glass lenses) and can be easily replaced when worn out. For example, the fixed shade lens 104 may be constructed of polycarbonate. The fixed shade lens 104 may be injection molded or stamped from a sheet of plastic and cut into the appropriate dimensions. The fixed shade lens 104 provides different shade levels or tinting. The shaded polycarbonate interior and/or exterior cover lenses can be made to any shade level, such as shade levels defined by American National Standards Institute (ANSI) standard ANSI Z87.1 and/or European Commission (CE) standards, in any increments. Construction of the fixed shade lens 104 is not limited to polycarbonate. While polycarbonate may be used for its excellent mechanical properties, acrylic, polystyrene, and/or blend of various plastic materials may also be used.

When installed into the head worn device 106, the fixed shade lens 104 cooperates with the variable shade component 102 to provide a net shade range. In some examples, the net shade value range has an upper shade value of at least 8.0. In some examples, the fixed shade lens 104 has a shade value greater than 0.0. In other examples, the fixed shade value is at least 0.1. In some such examples, the fixed shade value is at least 0.5. In some examples, the fixed shade value is at least 1.0.

The variable shade component 102 may have a lower limit of the shade value range of at least a 1.0 shade level. In some examples, the variable shade component 102 has a lower limit of the shade value range of at least a 2.5 shade level.

The example head worn device 100 also includes a clear lens 108 on an exterior of the variable shade component 102 and the fixed shade lens 104. The clear lens 108 has a shade level of substantially zero, and protects the variable shade component 102 from welding conditions, such as heat and spatter. The clear lens 108 is replaceable so that the clear lens 108 can be exchanged in the event of damage or wear.

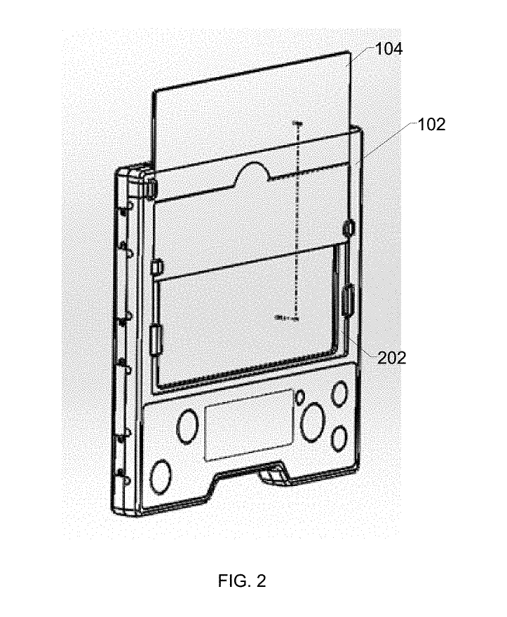

FIG. 2 illustrates installation of the fixed shade lens 104 of FIG. 1 into a lens retention device 202. The head worn device 106 includes a lens retention device 202 configured to releasably hold the fixed shade lens 104. The fixed shade lens 104 may be inserted or removed from the lens retention device 202, which enables different fixed shade lenses to be used to result in different net shade ranges for the head worn device 106.

The lens retention device 202 may be a slot, a clip, or any other type of retention device for the fixed shade lens 104. The lens retention device 202 may be located on an interior side of the variable shade component 102 or an exterior side of the variable shade component 102.

In some other examples, the fixed shade lens 104 is affixed or adhered to an interior or an exterior of the variable shade component 102. For example, the fixed shade lens 104 may be a decal that is affixed to the variable shade component 102, and which may be removed to affix another fixed shade lens with a different shade value.

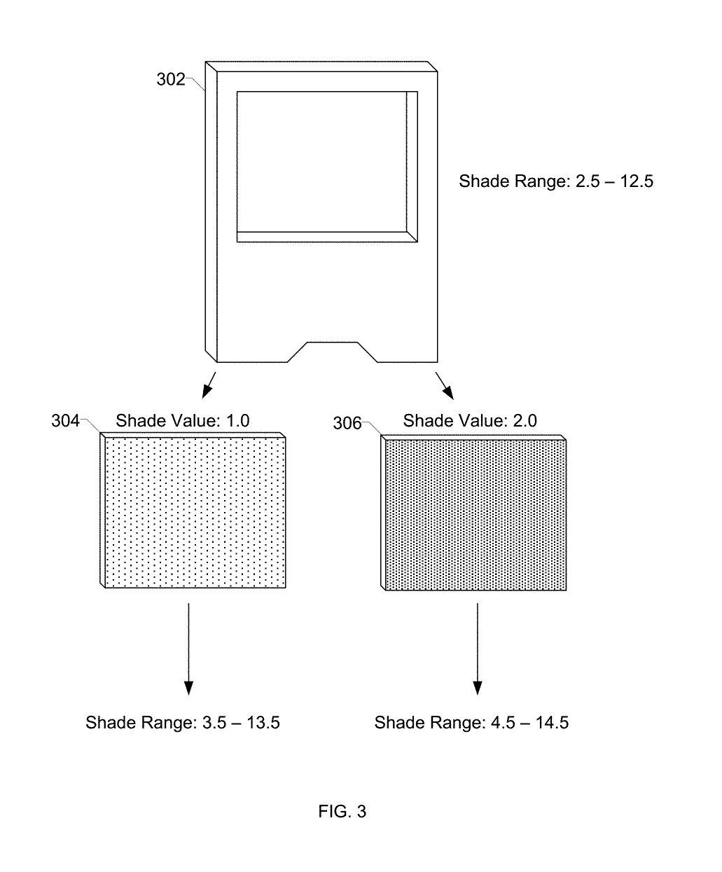

FIG. 3 illustrates combinations of an auto-darkening lens 302 and fixed shade lenses 304, 306 and resulting net shade value ranges. The auto-darkening lens 302 of FIG. 3 has an example shade range of 2.5-12.5. That is, the auto-darkening lens 302 may change its shade value between 2.5 (e.g., when there is relatively little ambient light, such as when there is no welding arc present) and 12.5 (e.g.., when there is a high level of ambient light present, such as when an intense welding arc is present).

A first fixed shade lens 304 has a fixed shade value of 1.0. When the first fixed shade lens 304 is installed into a lens retention device (e.g., a slot or clip), the combination of the shade range and the fixed shade value of the first fixed shade lens 304 combines to result in a net shade range of 3.5-13.5.

A second fixed shade lens 306 has a fixed shade value of 2.0. When the second fixed shade lens 306 is installed into a lens retention device (e.g., a slot or clip), the combination of the shade range and the fixed shade value of the second fixed shade lens 306 combines to result in a net shade range of 4.5-14.5.

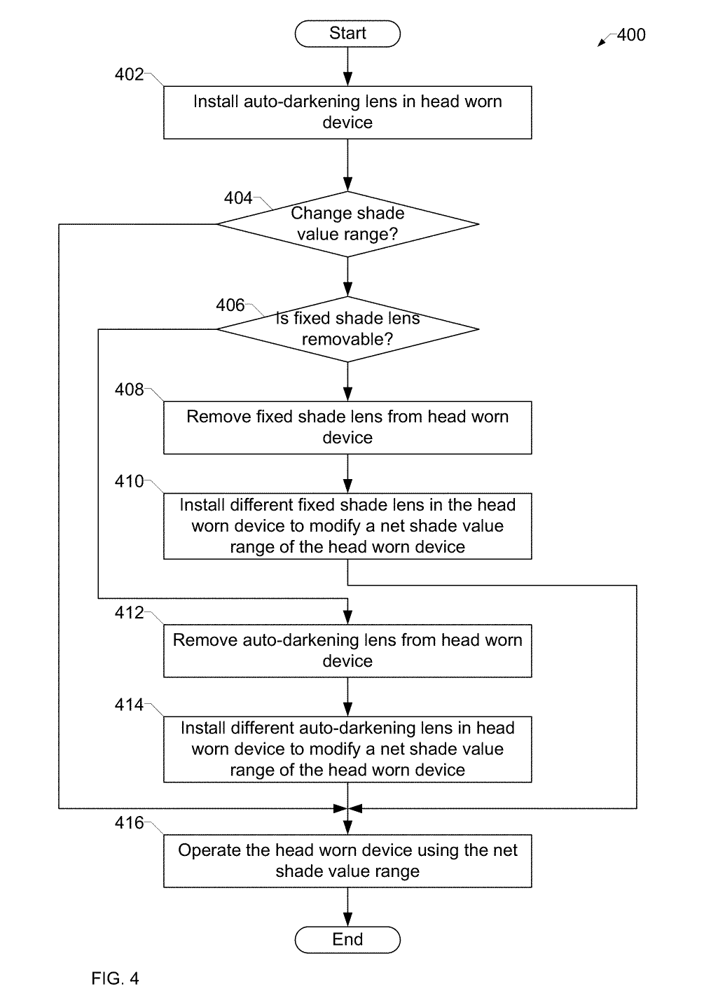

FIG. 4 is a flowchart representative of a method 400 to operate the example head worn device 106 of FIG. 1.

At block 402, the operator installs an auto-darkening lens (e.g., the auto-darkening lens 102 of FIG. 1) in a head worn device (e.g., the head worn device 106 of FIG. 1). For example, an operator may select an auto-darkening lens having a desired shade range value, and install the auto-darkening lens into the head worn device 106.

At block 404, the operator decides whether to change a shade value range. For example, the operator may decide that a different net shade value range than a current net shade value range of the head worn device 106 is desired. If the shade value range is to be changed (block 404) and the fixed shade lens 104 is removable (block 406), at block 408 the operator removes an installed fixed shade lens 104 from the head worn device 106. For example, the operator may remove the fixed shade lens 104 from a lens retention device (e.g., the lens retention device 202 of FIG. 2).

At block 410, the operator installs a different fixed shade lens in the head worn device 106 (e.g., in the lens retention device 202) to modify the net shade value range of the head worn device 106. For example, the operator may increase a net shade range of the head worn device 106 by removing the fixed shade lens 304 of FIG. 3 and installing the fixed shade lens 306.

If the shade value range is to be changed (block 404) and the fixed shade lens 104 is not removable (i.e., the fixed shade lens 104 is installed) (block 406), at block 412 the operator removes the auto-darkening lens 102 from the head worn device 106. At block 414 the operator installs a different auto-darkening lens 102 in the head worn device to modify a net shade value range of the head worn device 106. For example, the operator may decrease a net shade value range for a given fixed shade lens 104 by replacing an auto-darkening lens having a shade range of 3.0-13.0 with an auto-darkening lens having a shade range of 2.5-12.5.

After installing a different auto-darkening lens (block 414), after installing a different fixed shade lens (block 410), or if the shade value range is be maintained (block 404), at block 416 the operator operates the head worn device using the net shade value range.

After operating the head worn device (block 416), the example method 400 may end.

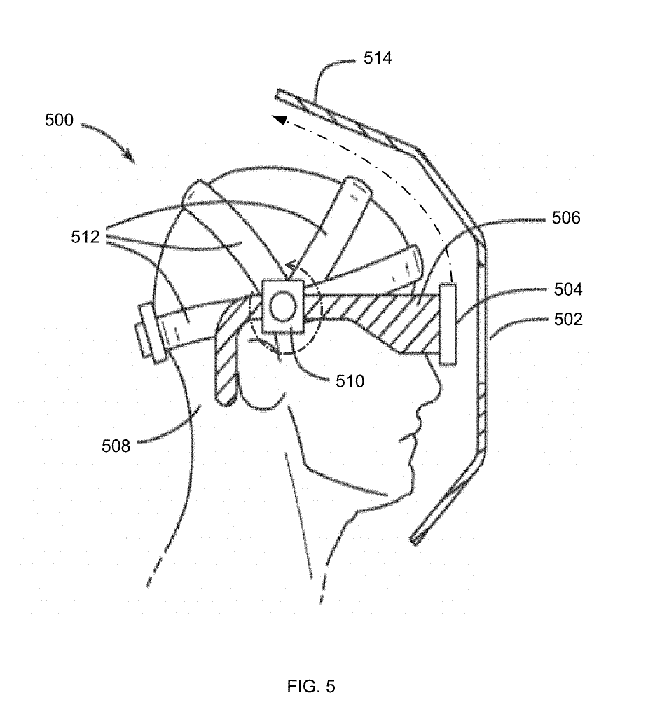

FIG. 5 illustrates another example head worn device 500 in which a fixed shade lens 502 may be moved into and at least partially out of a field of view of a variable shade lens 504.

The fixed shade lens 502 is located on the interior of a second, auto darkening lens 504 in the illustrated configuration. The fixed shade lens 502 is attached to a frame 506 and can pivot in and out of the field of view of a wearer 508 by a pivot mechanism 510 where a frame 506 is coupled to the headgear 512. The frame 506 can be telescopic to account for different user head shape and size. The rotation of the fixed shade lens 502 occurs on the interior of a shell 514.

The example head worn device 500 of FIG. 5 provides the wearer 508 with a lighter light state (e.g., non-welding state) by permitting the user to move the fixed shade lens 502 in and out of their field of view. Such a head worn device 500 may reduce the likelihood that the wearer 508 chooses to lift up the entire head worn device 500 (e.g., as is sometimes done with conventional welding helmets that have light states that are too dark for ambient lighting) and thereby expose the wearer 508 to eye injuries. The head worn device 500 provides the wearer 508 with modularity between shade values that meet the wearer's 508 needs, thereby reducing the need to lift up the head worn device 500. Thus, the head worn device 500 can improve the safety of welders.

Alternatively, the fixed shade lens 502 may be paired with a second fixed shaded type, where the fixed shade lens 502 has a light filtering value and, when overlapping with a second fixed shaded lens with a second shade value, results in a third fixed shade value. In still other examples, the fixed shade lens 502 is replaced could be used to a similar effect is if the fixed shade lens 502 is replaced with an auto-darkening lens and the auto darkening lens 504 is replaced with a fixed shade lens.

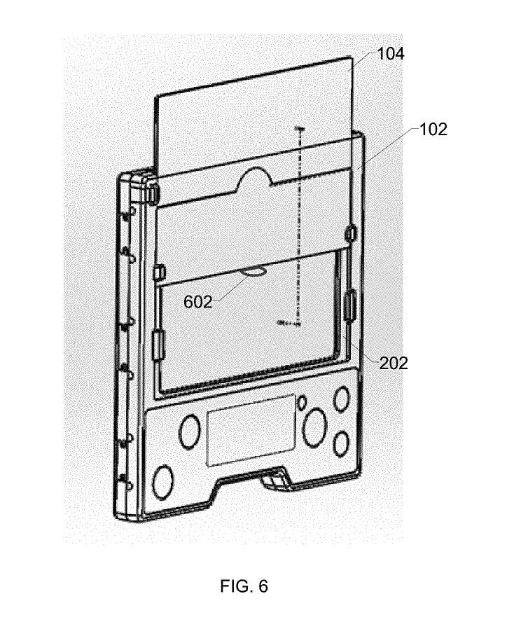

FIG. 6 illustrates another example installation of the fixed shade lens 104 of FIG. 1 into a lens retention device 202. In the example of FIG. 6, the fixed shade lens 104 is fitted into a rail mechanism 202 located on the interior surface of the auto-darkening lens 102. The fixed shade lens 104 includes a lift tab 602 that allows a user to easily slide the fixed shade lens 104 into the field of view of the auto-darkening lens 102 and at least partially out of the field of view of the auto-darkening lens 102. The lift tab 602 allows the fixed shade lens 104 to be placed in a position where the fixed shade lens 104: overlaps with the auto-darkening lens 102 to create a third shade value; does not overlap with the auto-darkening lens 102 to provide the welder with the shade value or range of only the auto-darkening lens 102, and/or a position in which a portion of the fixed shade lens 104 overlaps with the auto-darkening lens 102.

Other configurations and/or mechanisms to move the fixed shade lens 104 to be moved into and out of the field of view of the welder. For example, the movement mechanism could be located on the welding helmet shell surface.

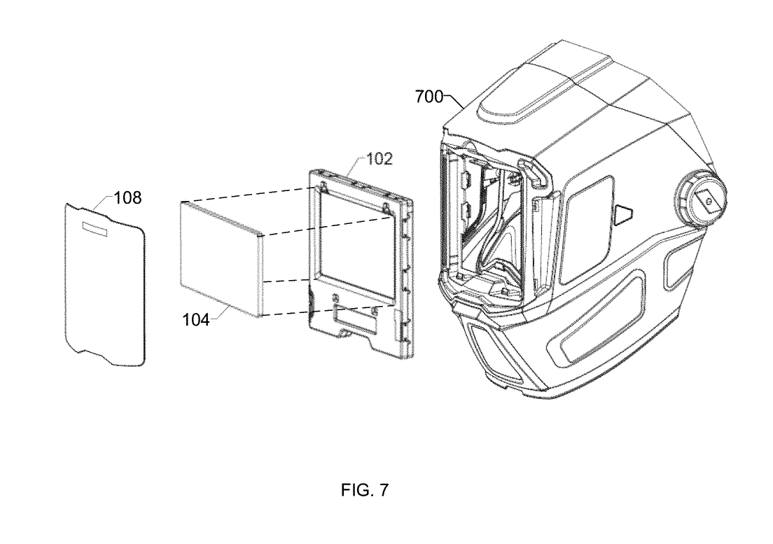

FIG. 7 illustrates another example combination of the variable shade component 102 and the fixed shade component 104 for use in a head worn device 700. The example head worn device 700 is similar to the head worn device 106 of FIG. 1, except that the fixed shade lens 104 is located on an exterior of the variable shade lens 102. For example, the fixed shade lens 104 may fit into a slot on an exterior side of the variable shade lens 102. The clear lens 108 is located on the exterior of both the fixed shade lens 104 and the variable shade lens 102.

As utilized herein, “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”. As utilized herein, the term “exemplary” means serving as a non-limiting example, instance, or illustration. As utilized herein, the terms “e.g.,” and “for example” set off lists of one or more non-limiting examples, instances, or illustrations.

While the present method and/or system has been described with reference to certain implementations, it will be understood by those skilled in the art that various changes may be made and equivalents may be substituted without departing from the scope of the present method and/or system. In addition, many modifications may be made to adapt a particular situation or material to the teachings of the present disclosure without departing from its scope. For example, blocks and/or components of disclosed examples may be combined, divided, re-arranged, and/or otherwise modified. Therefore, it is intended that the present method and/or system not be limited to the particular implementations disclosed, but that the present method and/or system will include all implementations falling within the scope of the appended claims, both literally and under the doctrine of equivalents.

What is claimed is:

1. A head worn device, comprising:
an auto-darkening lens configured to adjust a shade value within a first shade value range;
a second lens having a fixed shade value greater than 0, the auto-darkening lens and the second lens overlapping in a field of view of the head worn device to result in a net shade value range in the field of view that is greater than the first shade value range; and
a clear lens on an exterior of the auto-darkening lens and the second lens.

2. The head worn device as defined in claim 1, further comprising a lens retention device configured to releasably hold the second lens.

3. The head worn device as defined in claim 2, wherein the lens retention device is configured to hold a third lens having a second fixed shade value.

4. The head worn device as defined in claim 3, wherein the second fixed shade value is between 0.0 and 1.0.

5. The head worn device as defined in claim 2, wherein the lens retention device comprises a slot or a clip on an interior side of the auto-darkening lens.

6. The head worn device as defined in claim 2, wherein the lens retention device is located at an exterior of the auto-darkening lens.

7. The head worn device as defined in claim 1, wherein the fixed shade value is at least 0.1.

8. The head worn device as defined in claim 1, wherein the fixed shade value is at least 0.5.

9. The head worn device as defined in claim 1, wherein the fixed shade value is at least 1.0.

10. The head worn device as defined in claim 1, wherein the first shade value range has a lower limit of at least a 1.0 shade level.

11. The head worn device as defined in claim 1, wherein the net shade value range has an upper shade value of at least 8.0.

12. The head worn device as defined in claim 1, wherein the auto-darkening lens is configured to adjust the shade value within the first shade value range based on an ambient light level or a received signal.

13. The head worn device as defined in claim 1, wherein the second lens is removably affixed to the auto-darkening lens.

14. The head worn device as defined in claim 1, wherein the second lens is integral to the head worn device, and the auto-darkening lens is configured to be attached and detached from the head worn device.

15. The head worn device as defined in claim 1, further comprising a lens retention device configured to permit movement of the second lens into the field of view and at least partially out of the field of view.

16. A method, comprising:
installing an auto-darkening lens in a head worn device, the auto-darkening lens configured to change a first shade value within a first shade value range;
installing a clear lens on an exterior of the auto-darkening lens and a first fixed shade lens having a shade value greater than 0 within a field of view of the head worn device; and
operating the head worn device using a second shade value range based on the first shade value range and the first fixed shade lens on the head worn device.

17. The method as defined in claim 16, further comprising installing the first fixed shade lens on the head worn device, the first fixed shade lens having a first fixed shade value which, when installed, modifies the first shade value range to the second shade value range.

18. The method as defined in claim 17, further comprising removing a second fixed shade lens from the head worn device.

19. The method as defined in claim 16, wherein the installing of the first fixed shade lens comprises inserting the first fixed shade lens into a lens retention device of the head worn device.

20. The method as defined in claim 16, wherein the installing of the first fixed shade lens comprises attaching the first fixed shade lens to the auto-darkening lens.

21. The method as defined in claim 16, further comprising performing a welding operation, the welding operation causing the head worn device to modify the first shade value within the first shade value range.

22. The method as defined in claim 16, further comprising moving the first fixed shade lens at least partially out of the field of view of the head worn device using a lens retention device.
